(19) United States
(12) Patent Application Publication
(10) Pub. No.: US 2013/0301007 A1
(43) Pub. Date: Nov. 14, 2013

(54) APPARATUS TO MEASURE ACCOMMODATION OF THE EYE

(71) Applicants: James Wolffsohn, West Midlands (GB); Mark Prince, West Midlands (GB); James Watkins, West Midlands (GB)

(72) Inventors: James Wolffsohn, West Midlands (GB); Mark Prince, West Midlands (GB); James Watkins, West Midlands (GB)

(73) Assignee: Aston University, Birmingham (GB)

(21) Appl. No.: 13/822,866

(22) PCT Filed: Sep. 14, 2011
(86) PCT No.: PCT/GB2011/051719

§ 371 Date: Jun. 4, 2013

(30) Foreign Application Priority Data
Sep. 14, 2010 (GB) 1015282,5

(51) Int. Cl. A61B 3/09

(52) U.S. Cl. CPC A61B 3/09
USPC 351/239; 351/246

(57) ABSTRACT

A device for measuring a user's range of clear focus (accommodation) comprising: a display for displaying a test image to the user at a first size; a sensor for determining the separation between the display and the user; and a processor for determining the angular subtence of the image to the user and to resize the test image in order to substantially maintain the angular subtence of the image when the separation between the display and the user is varied.

### TECHNICAL FIELD

The present invention relates to apparatus for measuring the range of clear focus of the eye. In particular, the invention related to a device, preferably portable, that can compared an individual's range of accommodation to that expected for their age to identify the need for intervention.

### BACKGROUND TO THE INVENTION

The range of distances at which an eye, such as a human eye, can clearly focus on an object is known as the accommodation of the eye.

Accommodation (the ability to focus) is due to the ability of the ciliary muscle to contract, slackening the zonular fibres which attach to the elastic crystalline lens inside the eye. This allows the lens to take a more spherical shape, becoming more optically powerful and focusing light rays from near objects on the back of the eye where the light receptors are located.

The amplitude of accommodation (or range of clear focus) decreases with age from about the age of 10 years. The amplitude of accommodation is defined as the distance between the nearest and furthest points at which the eye can focus on an object, usually expressed in dioptres (1/distance in metres).

Accommodation is typically measured by a RAF rule also known as an accommodometer. The RAF rule comprises a metal rod, which is rested against a user's nose or face and a display mounted onto the rod. The display contains a target image on which the user must focus. The display is progressively moved towards the user who then indicates when the target image on the display is no longer in focus; the point of “first blur” where the target is no longer resolved clearly by the user. When the image is no longer in focus, the distance of the display from the user is measured, allowing for the determination of the amplitude of accommodation from which the eye age of the eye may be determined (see, for example, Hamasaki et al. 1956, American Journal of Optometry and Archive of American Academy of Optometry).

The RAF rule is large and cumbersome and as the user is required to rest their nose or face on the rule, some people find the physical nature of the device off-putting. It is also known that the RAF rule can overestimate accommodation, as when the target approaches the user more blur can be tolerated and the pupil size is also affected, changing the depth of focus.

To mitigate at least some of the problems in the prior art there is provided a device for measuring near field vision comprising: a display for displaying a test image to a user at a first size; means for determining the separation between the display and the user such as a sensor; and preferably a processor for determining the angular subtence of the image to the user and to resize the test image in order to substantially maintain the angular subtence of the image when the separation between the display and the user is varied.

Preferably the device is handheld, and the sensor is a contactless sensor such as an ultrasonic transducer. Preferably the device further comprises a user input device, such as touch screen, buttons. Preferably wherein the device further comprises a wireless communicator enabled to transmit and receive data from an external source, and a memory. Preferably wherein the device is further enabled to determine the eye age of the user, and the memory comprising a table or equation of eye age versus diopter thereby allowing determination of eye age. Preferably wherein in use calculation of distance and image size occur at a rate greater than 16 times a second preferably greater than 24. Preferably the device has one or more additional sensors, preferably including speed sensor and attitude sensor, and/or alignment means to prevent off-axis viewing, such as a privacy shield. Optionally wherein the display further comprises a parallax barrier to provide a 3D image on the display, and wherein the septums of the parallax barrier are resized in use to maintain a 3D image when the distance between the use and device is changed.

There is also provided a method for determining near field vision the method comprising: displaying a test image of a first size on a display; measuring the separation of a user from the display; determining the angular subtence of the image; and resizing the image on the display when the separation between the user and the display has changed so that the angular subtence of the image remains substantially constant.

Preferably the method further comprises the step of: the user indicating via a user input that the test image has become unclear, and further comprises the step of measuring the diopter of the eye and calculating the eye age of the user.

There is also provided a computer readable product containing instructions thereon to implement the above method.

Other aspects of the invention will be clear from the appended claim set.

Further aspects, features and advantages of the present invention will be apparent from the following description and appended claims.

### BRIEF DESCRIPTION OF THE DRAWINGS

An embodiment of the invention will now be described by way of example only, with reference to the following drawings, in which:

FIG. 1 is a plan view of the front of apparatus according to an embodiment of the invention;

FIG. 2 is a plan view of the rear of the apparatus of the embodiment of FIG. 1;

FIG. 3 is a schematic representation of the apparatus;

FIG. 4 is a flow diagram of a process of determining the accommodation of the eye;

FIG. 5 schematically shows a process of resizing the image on the display;

FIG. 6 is a plan view of the front of the apparatus according to a smartphone implemented embodiment of the invention; and

FIG. 7 is a schematic representation of a process for determining separation according to an aspect of the invention.

### DETAILED DESCRIPTION

There is provided a device, preferably handheld, which can display a target test image on a screen. The device is enabled to determine the distance/separation of the screen from a user's eyes, and the angular subtence of the image adjusted initially for the patient's visual resolution is then maintained. Angular subtence is the angle of the object viewed as measured from the entrance of the pupil of the eye. As the user moves the device closer from their eye, if the image is of a fixed size, the angular subtence of the image will increase. Similarly if the device is moved away from the eye the angular subtence decreases. This variation in angular subtence is undesirable as it introduces inaccuracies in the accommodation measurement as a larger level of blurring is acceptable for a large object than for a small object, accordingly the point of “first blur” may be inaccurately measured. Therefore, the device is further enabled to resize the image shown on the screen so that the image presented to the user is at a constant angular subtence.

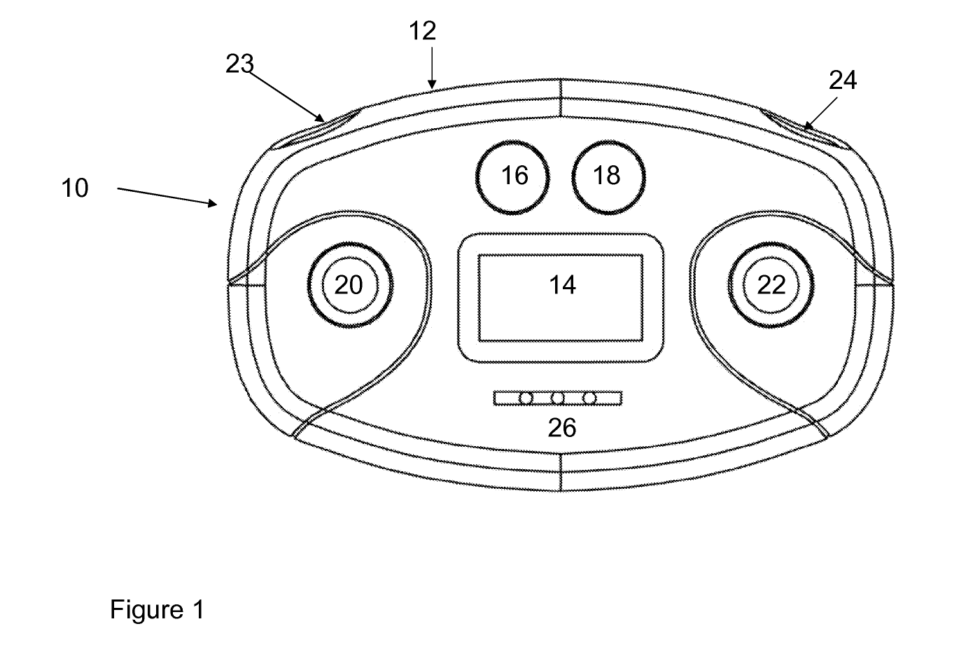

FIG. 1 shows an example of the apparatus according to an embodiment of the invention.

There is shown: device 10 having a housing 12; display 14; ultrasonic sensors 16, 18; user input buttons 20, 22, 23 and 24 and LEDs 26.

The device 10 is a handheld device which is operated by a user (in this case a patient who is having their near field focusing range determined). The device 10 is contained within a housing 12 and is configured to be handheld and of comparable size to a mobile telephone device or handheld games console. The housing 12 is typically a thermoplastic, which can provide protection against minor impacts, though other suitable materials may be used. The housing contains a display 14, typically an LCD or OLED display though other types of display can be used. The display 14 can show a number of different images and can resize an image shown on the display.

The device 10 further comprises one or more sensors 16 18 which are stored in the housing 12. In the embodiment shown, there are two ultrasonic sensors or transducers 16 18 which are used to determine the distance of the device 10, and therefore the display 14, from a user. The sensors 16 18 are a combination of emitter and receivers and may be commercially available ultrasonic sensors. Such sensors are preferred due to their high accuracy and low manufacturing cost. In further embodiments, other types of contactless sensors may be used.

The device 10 further contains user input buttons 20 22 23 and 24 which a user can actuate with the users fingers or thumbs. There are also LED lights 26 which indicate to the user when the device 10 is powered up and preferably when a test is taking place.

The housing 12 further contains a power source, processor, memory and wireless communication means (not shown in FIG. 1).

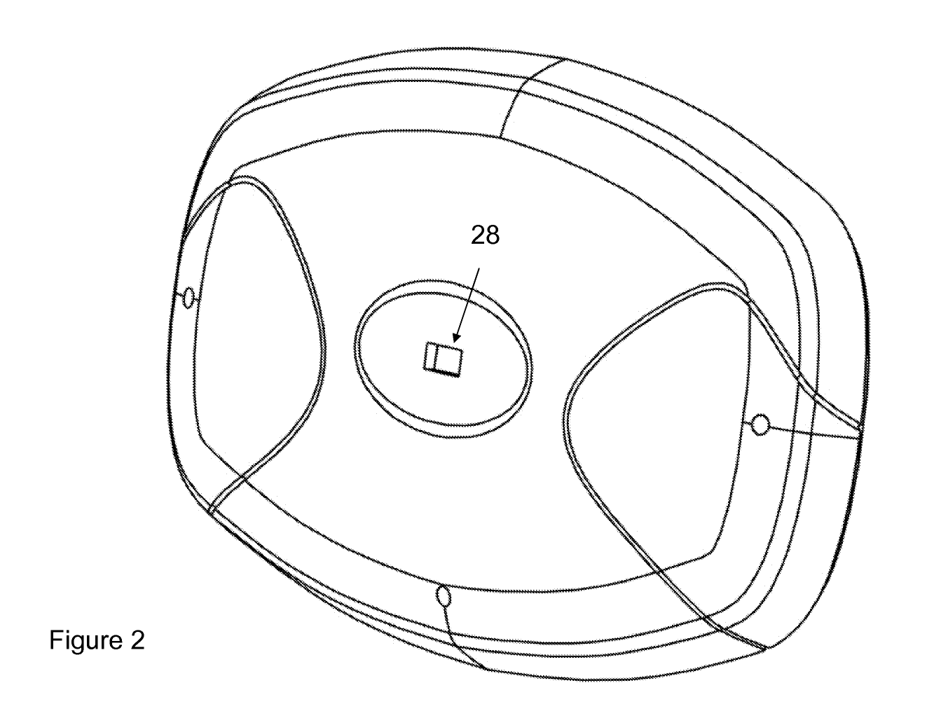

FIG. 2 is a rear view of the apparatus of FIG. 1. The device further comprises an on/off switch 28 and charging port (not shown) which preferably is also a USB port to allow wired communication with a computer (not shown). This port may also be used to charge a rechargeable power source to power the device 10.

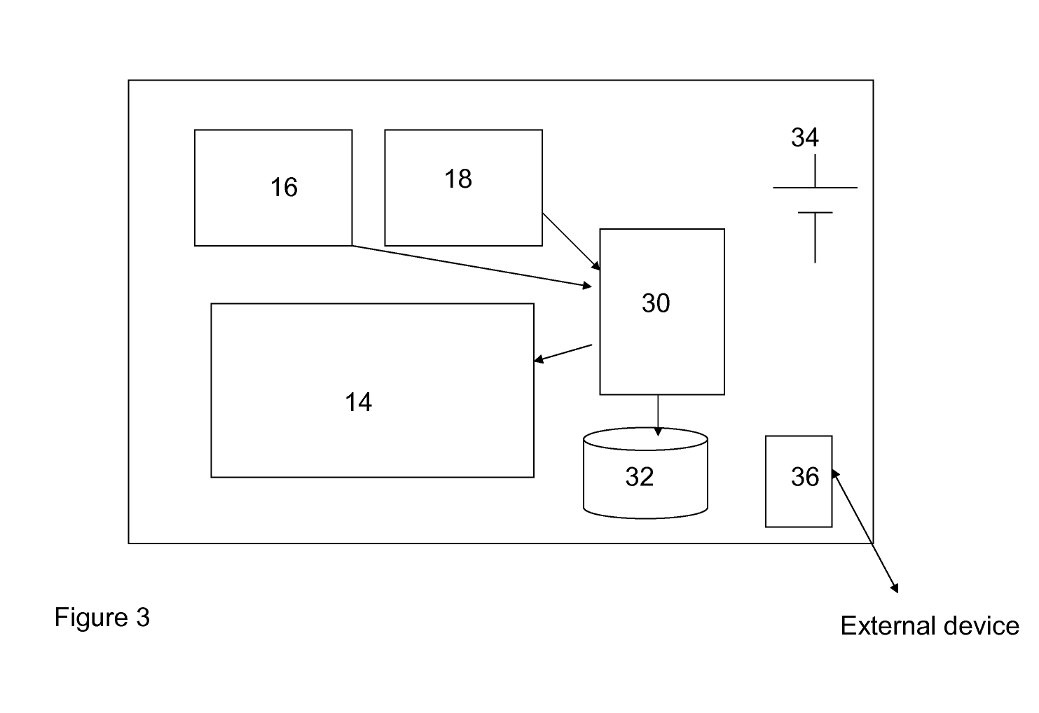

FIG. 3 is a schematic representation of the apparatus such as device 10 within the housing 12. There is shown: housing 12 containing the display 14; sensors 16 18; the sensors in communication with a processor 30; memory 32; power source 34 and wireless communication means 36.

The sensors 16 18 are in communication with a processor 30. In use the ultrasonic sensors 16 18 emit the ultrasonic signal which is reflected from the face of the user and detected. The detected signal is passed to the processor 30 which determines the distance of the user from the display using known distance determination techniques.

Such detectors 16 18 and processor 30 may be known commercially available products. The ultrasonic detectors typically allow for an accuracy in the measurement of the distance to a user of ±1 mm or less and determine a distance based on the measured delay between emission and receipt of an ultrasonic signal or pulse.

In further embodiments, other forms of sensor are used to measure distance are used, for example laser, potentiometer, infrared etc.

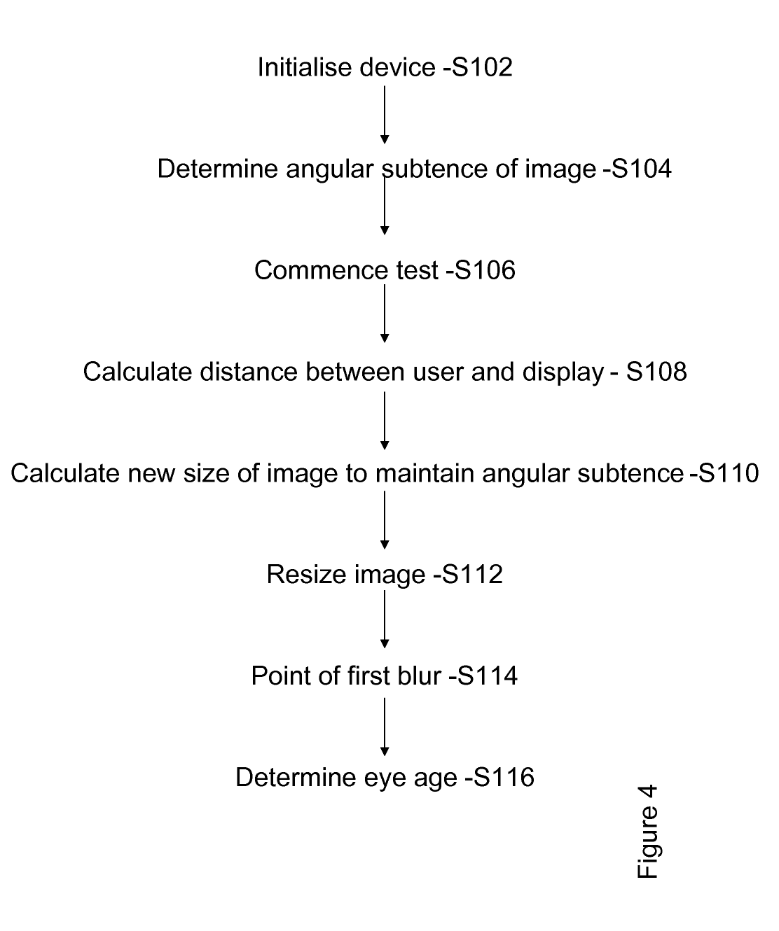

The processor 30 is configured to drive the display 14 so that the target image is presented at a constant angular subtence to the user regardless of the distance of the user from the display (this process is discussed in detail with reference to FIG. 4). Optionally, the processor 30 can write the measured distances and other information to a memory 32. The housing 12 also, preferably, contains a wireless communication means 36 which complies with the 802.11 standards, though other wireless means e.g. short range radio, GPRS etc. may be used.

The power source 34 powers the device. In a preferred embodiment, the power source 34 is a 9V power source stepped down to 5V. Preferably, the power source 34 is a rechargeable power source, such as a NiCd, NiMH, Li-ion battery. In a further, less preferred embodiment, the device 10 is powered through a wired connection via the charging port.

FIG. 4 shows a process of measuring accommodation according to an aspect of the invention.

At step S102 the device 10 is initialised. The device 10 is switched on and a target image is selected to be displayed on the display 14. Preferably, the initialising step S102 is performed by the administrator of the test, who is typically an optometrist, optician or technician.

The administrator may select the test image to be viewed from a separate computing device which communicates with the device 10 via the wireless communication means 36. The image selected may be any suitable test image, for example, a cross, a letter etc. It is found that for young children an image of a clown's face an animal, or the like, can beneficially be used to maintain a child's attention. In another embodiment the user may select the test image by actuating user input button 20 which displays a different test image on the display 14 to make their selection.

In a preferred embodiment, the device 10 is held at arm's length by the user and the size of the test image is varied until the smallest threshold image is reached i.e. the minimum size the test image can be before the user is unable to resolve the image. When the test image is at the minimum size the distance of “first blur” can be more accurately determined, as due to the small size of the image even a small amount of blurring is noticed by the user, whereas a similar amount of blurring for a larger image may not be noticed. In other, less preferred embodiments, larger images may be used.

The test image may be varied in size by the administrator communicating to the device via the wireless communication means 36.

In a further embodiment, at step S102, the user is presented with an interface on the display 14. Through the graphical user interface the user can select the image (e.g. letter, cross, clown face etc.) and vary the size of the image through the user input buttons 23 24 which increase and decrease the size of the image on the display 14. In further embodiments, the display 14 is a touch screen and the user can make their selection via the touch screen, via touch screen inputs or gesture based inputs.

When the test image has been selected the angular size of the image, measured as the angular subtence, is determined at step S104. The sensors 16 18 are activated and a distance from the user to the display 14 is determined. As the individual pixel size of the display 14 is known and the pixel size of the test image is known (from the initialising step S102) the physical size of the image on the display 14 can be determined. Using the measured distance of the display to the user (D) and the actual size of the image (d) the angular subtence of the image (δ) is calculated as:

δ=2 arctan(d/2D)

In further embodiments corrections may be made to take into account the off-set of the sensor from the display, the angle of the device, errors in the distance determination etc.

At step S106 the test begins and the user or assessor moves the device 10 closer to the user's eyes.

At step S108 the sensors 16 18 calculate the distance/separation between the display and the user (D).

Using the new value of distance (D) and keeping the constant angular subtence of the image (6) the size of the image (d) needed to maintain the constant angular subtence is calculated at step S110. i.e. the new value of distance (D) is used to calculate a new image size (d) for a constant value of δ.

Once the new size of the image is calculated at step S112 the processor 30 resizes the test image on the display 14. Therefore during the test the processor maintains a constant angular subtence of the image on the display 14.

Steps S110 and S112 are constantly reiterated during the test. Preferably, the image is resized at a rate faster than the flicker fusion threshold of the eye so that the user does not notice the resizing of the test image. Therefore, steps S110 and S112 are preferably repeated more than 16 times per second, more preferably around 24 times per second.

At step S114 the user has determined the image has become blurred. The user can indicate the point at which “first blur” i.e. when the image is no longer resolved, by actuating the user input buttons 20 22. In alternative embodiments the user indicates to the administrator that the image has become blurred e.g. by verbally informing the administrator.

When the user presses the user input button 22 the distance of the device 10 from the user is measured and is taken to be the nearest point at which the user can focus.

If desired the user can refine the measurement by moving the device 10 and taking multiple measurements to determine the first blur point.

Once the point of first blur has been determined, the diopter of the eye can be determined and compared with the typical age at which that level of accommodation remains at step S116. Preferably, the ‘eye age’ is calculated by the processor 30 using predetermined tables or equations of diopter versus ‘eye age’, therefore removing the need for the administrator to calculate the ‘eye age’. The ‘eye age’ can be displayed on the display 14 or on the computer of the administrator of the test. Optionally, the determined ‘eye age’ is also written to the memory 32.

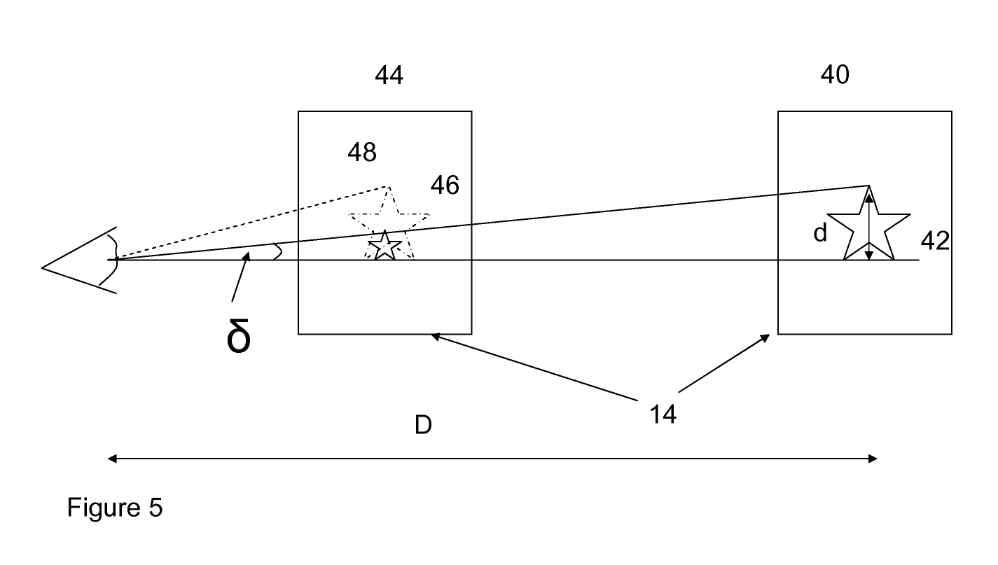

FIG. 5 shows a schematic diagram of the resizing of an image so that the angular subtence is maintained.

The display 14 is shown at two different distances D, the initial position 40, and a secondary positions 44. The target image 42 is represented as a star.

At the initial position 40, the test image 42 has a height d and the angular subtence δ is calculated (as per step S 104). As the display is moved to the secondary position 44 it is clear that in order to maintain the angular subtence the height d of the image 42 must be reduced. If the same size of star 42 where used at secondary position 44 as for the initial position 42 it is clear that the angular subtence δ would increase.

The dotted star 46 at the secondary position 44 is the size of the test image 42 at the initial position 40 and the dotted line 48 represents what would be the angular subtence if the test image had not been resized. It is clear that the angular subtence δ in the secondary position has increased.

If the test image is asymmetric the angular subtence in the x and y direction δₓ and δ_{y} is calculated at the initial position (i.e. as per step S104) and the subsequent positions (step S110). The image is then rescaled in both the x and y directions accordingly, thereby maintaining the aspect ratio of the original image as well as the angular subtence.

It will be appreciated that other embodiments beyond those described above can also be used. For example, in an embodiment the size of the image is set by the user, via the user input buttons 20 22 (or any other suitable means) to resize the image until a suitable size of image is displayed.

In further embodiments, the device 10 comprises further sensors such as speed and attitude sensors. If the device 10 is determined to be moving in excess of predetermined threshold, which would potentially lead to inaccurate measurements, the user is informed, for example via a message on the device or an audible alarm, to indicate to the user that the speed at which they are performing the test is greater than would be expected. In an embodiment with an attitude sensor, the sensor is used to ensure that the device is held in an upright position. If the processor 30 determines the device 10 is at an angle, the user is informed as described above. In a further embodiment a screen privacy filter is placed on top of the display 14, which prevents off-axis viewing of the screen thereby ensuring that the device 10 is viewed at the correct angle.

Optionally, the sensors can also be used to determine if the device 10 is shaking which would potentially lead to inaccurate results. If the device 10 is used by the very young or infirm the device 10 may be supported by a guide means. The guide means help prevent problems with shaking, with the user enabled to bring the device 10 closer to their eyes in a controlled manner.

In a further embodiment the display 14 has a parallax barrier to display the image in 3D. Such parallax barriers may be commercially available barriers. The parallax barrier is formed by a second overlaid screen, with the width of the spacings of the septums required to create the 3D effect being distance dependent. When the image presented is a 3D image, the distance measured (D) can be used to determine the separation of the septums to maintain the 3D effect when the device 10 is moved by the user. A further advantage of the parallax barrier is that off-axis viewing causes the 3D effect to disappear thereby ensuring the user does not view the screen at an angle.

The test displayed may also vary. In an embodiment, a reading test is displayed on the screen with the size of the text varying to maintain a constant angular subtence, as described with reference to FIG. 4, when the distance between the user and display 14 is varied. Tests to assess the binocular function of the two eyes are also envisaged, using polarised screens and glasses to determine the alignment of the eyes or their ability to determine depth (stereopsis).

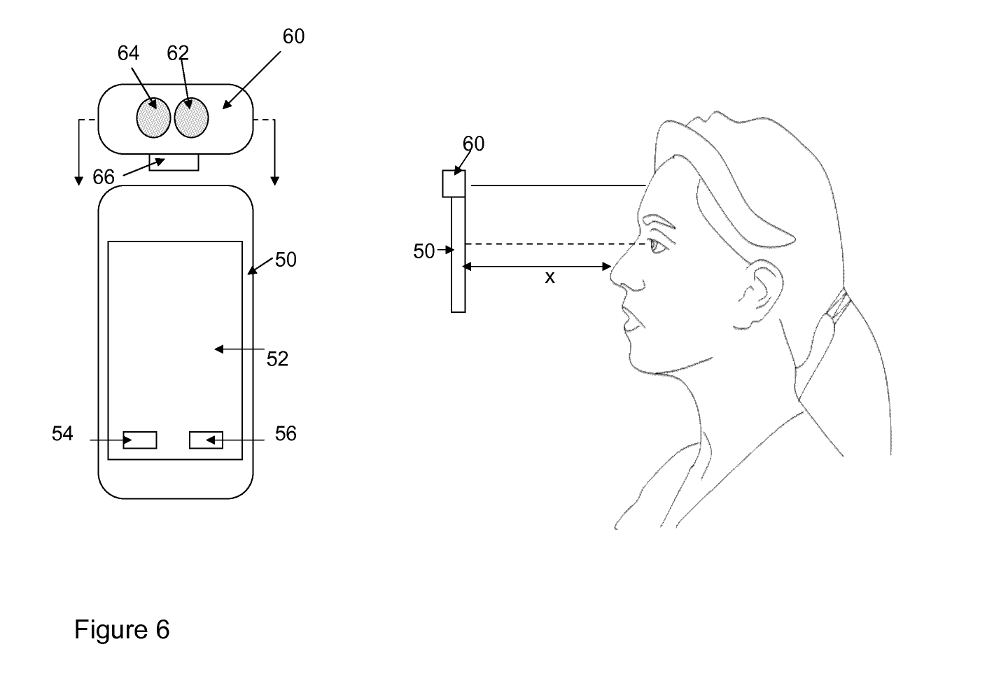

FIG. 6 shows a smartphone based embodiment of the invention.

In this embodiment the apparatus is incorporated as part of an existing smartphone device or known tablet device/computer. Such smartphones and tablet computers have one or more processors which can be used to run distributed software applications such as an “app”. Advantageously, the app uses the existing functionality of the smartphone or tablet device, such as existing screen, graphics and user interface technology, processor, display, memory, communication modules etc. This allows for the smartphone or tablet computer to be configured to provide further functionality that may not have been originally provided or indeed envisaged by the manufacturer of the original product. The following description is given with reference to a smartphone device, though it is noted that the embodiment discussed is applicable to other devices such as tablet computers, personal digital assistants (PDAs) etc.

In FIG. 6 there is shown the smartphone 50, display 52, buttons 54, 56, sensor attachment 60 having a combination of emitter and receiver 62 and 64 and connector to the smartphone 66.

The embodiment shown in FIG. 6 works using the same principles as described previously with reference to FIG. 4. In use, the user connects the sensor attachment 60 to the smartphone 50. Most smartphones do not have the functionality to determine the separation between a user and the display and therefore an attachment 60 is used to provide an ultrasonic emitter and receiver 62 and 64 (though in further embodiments other types of contactless sensor may be used). The attachment 60 has an embedded microcontroller (not shown) to drive and manage the sensors 62 64 in a known manner. The attachment 60 is attached to the smartphone 50 through known docking/serial ports e.g. USB, Firewire etc via the connection 66. Advantageously, attachment through an existing serial port and connection 66 allows for direct communication between the attachment 60 and the smartphone 50. Furthermore if the serial port allows for power transfer, in an embodiment the attachment 60 is powered through the serial port.

Alternatively can be attached to the body of the smartphone 50 via a clamp or clasp. The attachment 60 communicates with the smartphone via the known docking port or via near field communication protocols such as Bluetooth (RTM).

In use the attachment 60 initiates communication and authentication with the smartphone 50 via known protocols such as a handshake protocol. Once authenticated the process of determining accommodation as described with reference to FIG. 4 begins.

The user is presented with a test image, and holds the smartphone at arms' length and the attachment 60. The measurement made by the sensors 62 64 is communicated to the smartphone 50 and converted to a distance as described with reference to FIG. 4. The angular subtence of the image is also determined. As the smartphone is moved towards the user the sensors 62 64 measure the change in separation from the device to user and the size of the image presented on the display of the smartphone 50 is changed to maintain the angular subtence of the image. When the image reaches the “point of first blur” the user presses a button 54 56 to indicate the point of first blur has been reached.

Some smartphones 50 comprise buttons in the form of a keypad or with set functionality (not shown) and these buttons can be actuated to input to the smartphone 50. Other smartphones 50 do not have buttons (or only have a single button which is used to exit a program) and rely on a touchscreen interface. With such devices the buttons 54 56 are displayed on the touchscreen and the user presses the buttons on the touchscreen in the known manner.

In a further embodiment the smartphone does not use the ultrasonic sensor attachment 60 as shown in FIG. 6 but instead uses the camera that is typically found in such devices. It is known for smartphones 50 to have a “forward facing” camera which are positioned on the side of the same device as the display thereby allowing a user to film or take photos of themselves whilst viewing the display.

In such an embodiment the video camera embedded within the smart device is used to collect the measurement data by coupling the camera with a laser projector module. In the embodiment shown in FIG. 6, the attachment comprises ultrasonic transceivers. In a further embodiment the ultrasonic transceivers (or emitters and receivers) are replaced with a simple laser light source and diffractive lens. The diffractive lens is configured such that when the laser light source is emitting the user's face is overlaid with a preferably regular pattern of near-infra red features (dots, lines, boxes, arrows).

This pattern (along with the user's face) is imaged by the camera and identified by the processor using known pattern recognition techniques. As the shape of the feature (dots, lines etc.) is known the pattern can be easily recognised. As the light is emitted from a point source, and in the preferred embodiment the pattern is regular, the pattern which is projected on the face changes linearly in density with the separation between the laser projector/smartphone and the user's face. Therefore by measuring the separation of the features the distance between the smartphone and the user's face can be determined.

In further embodiments the sensor uses an existing depth sensor, which uses time of flight measurements from an infrared source to determine the separation between a user and a display.

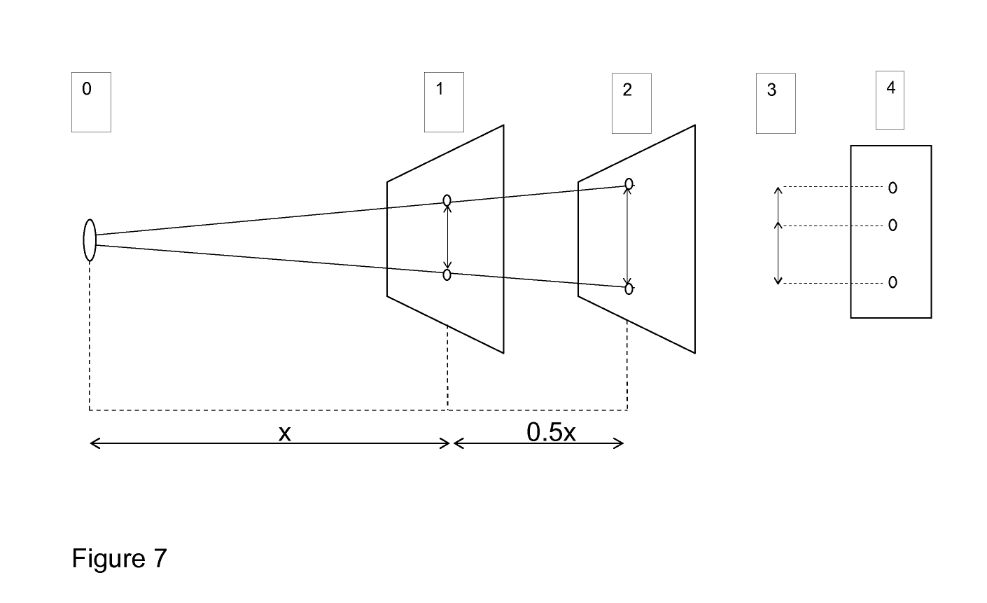

FIG. 7 schematically represents the change in the projected pattern according to the separation of the smartphone and the user's face.

Position 0 represents the projection source (i.e. the light source coupled to the diffractive lens which in an embodiment is placed on the smartphone). In the example shown the projected feature comprises two dots and the separation between the two dots are shown by the arrows. Position 1 represents a plane of projection (which would typically be a user's face but in the Figure is shown as a flat surface for simplicity) at some distance (x) from the projection source. Position 2 is another plane of projection which is 50% further from away from 0. The projected images at positions 1 and 2 are overlaid and shown at 3. As can be seen by the arrows the greater the distance the plane of projection is from the projection source, the further apart the dot features would appear (4).

If the image were also recorded at position 0, then the separation of the dots would appear to remain constant as the point of projection and imaging are the same. However the scale of the projection plane would differ with the plane at position 1 appearing 50% larger than that of position 2. The net result would be a higher pattern density on the nearer plane than on planes further away. This could be measured through a simple image counting algorithm, or through the application of an edge finding algorithm to determine facial disk size and to calculate projected feature density.

The software, or app, on the smartphone therefore comprises an image processing algorithm which compares the projected pattern as recorded by the camera with those of a pre-calibrated benchmark to determine the distance between the accommodometer apparatus (i.e. the smartphone) and the user. Advantageously the image processing apparatus can also be used to compare measurements across the disk of the face and return a measure of lateral alignment accuracy (based upon symmetry of facial disk) and thus ensure that the user's face is held in alignment with the smartphone. If the processor determines that the user's head is turned to one side or out of alignment, visual cues are presented on the display to the user to align their face. Furthermore the pattern can be used to return a more accurate average of the distance (based on using a number of measurements across entire facial disk).

The described embodiments therefore take advantage of existing functionality in smartphone and tablet devices and can also result in a lower production cost.

1. A device for measuring a user's range of clear focus (accommodation), the device comprising:
a display for displaying a test image to the user at a first size;
a sensor for determining the separation between the display and the user; and
a processor for determining the angular subtence of the image to the user and to resize the test image in order to substantially maintain the angular subtence of the image when the separation between the display and the user is varied.

2. The device of claim 1 wherein the device is handheld.

3. The device of claim 1 wherein the sensor is a contactless sensor or an ultrasonic transducer.

4. (canceled)

5. The device of claim 1 wherein the device further comprises a user input device, including touch screen buttons that accept user inputs.

6. (canceled)

7. The device of claim 1 wherein the device further comprises a memory and a wireless communicator enabled to transmit and receive data from an external source.

8. (canceled)

9. The device of claim 1 wherein the device is further enabled to determine the eye age of the user.

10. (canceled)

11. The device of claim 1 wherein in use calculation of distance and image size occur at least sixteen times a second preferably at least twenty four times a second.

12. The device of claim 1 further comprising one or more additional sensors, selected from the group of a speed sensor and attitude sensor.

13. The device of claim 1 further comprising alignment means to prevent off-axis viewing.

14. (canceled)

15. The device of claim 1 wherein the display further comprises a parallax barrier to provide a 3D image on the display, the parallax barrier comprising septums that are resized in use to maintain a 3D image when the distance between the user and device is changed.

16. (canceled)

17. The device of claim 1 wherein the device is a smartphone or tablet computer and the sensor for determining the separation is coupled to the smartphone or tablet computer.

18. The device of claim 17 wherein the sensor communicates with the processor of the smartphone or tablet computer using a near field communication protocol.

19. The device of claim 17 wherein the sensor communicates with the processor of the smartphone or tablet computer using a serial port present on the smartphone or tablet computer.

20. (canceled)

21. The device of claim 1 wherein the sensor is configured to project a predetermined pattern onto a surface and a camera to measure the projected pattern, wherein the processor is configured to determine the separation based on the measured pattern projected onto a surface, and wherein the processor is further configured to determine the alignment of a user's face based on the measured pattern.

22-25. (canceled)

26. A method for determining a user's range of clear focus the method comprising:
displaying a test image of a first size on a display;
measuring the separation of the user from the display;
determining the angular subtence of the image; and
resizing the image on the display when the separation between the user and the display has changed so that the angular subtence of the image remains substantially constant.

27. The method of claim 26 further comprising the step of:
the user indicating via a user input that the test image has become unclear.

28. The method of claim 26 further comprising the step of measuring the diopter of the eye and calculating the eye age of the user.

29. The method of claim 26 wherein the steps of measuring the separation comprises emitting and subsequently measuring an ultrasonic pulse and measuring the delay between emission and measurement of the pulse, and determining a separation based on the measured delay.

30. The method of claim 26 wherein the steps of measuring the separation comprises projecting a predetermined pattern onto a surface, measuring the pattern and determining a separation based on the measured pattern.

31. The method of claim 26 wherein the steps of measuring the separation comprises emitting an infrared source and subsequently measuring the time of flight of the flight and determining a separation based on the measured time of flight.

32. (canceled)
